(19) United States
(12) Patent Application Publication
(10) Pub. No.: US 2013/0226061 A1
(43) Pub. Date: Aug. 29, 2013

(54) MULTI-LAYER BANDAGES AND METHODS FOR DRESSING A WOUND

(71) Applicant: Richard M. Dickson, Kirkland, WA (US)

(72) Inventor: Richard M. Dickson, Kirkland, WA (US)

(21) Appl. No.: 13/543,699

(22) Filed: Jul. 6, 2012

Related U.S. Application Data
(60) Provisional application No. 61/634,501, filed on Feb. 29, 2012.
(60) Provisional application No. 61/686,539, filed on Apr. 5, 2012.

(51) Int. Cl. A61K 33/34; A61F 13/56; A61L 15/00; A61K 9/70; A61F 13/00

(52) U.S. Cl. 602/44; 424/449; 424/630; 602/48; 604/385.03

(57) ABSTRACT

Examples of multi-layer bandages and methods for dressing a wound are described. An example multi-layer bandage includes a first layer and second layer and an optional third layer. An exemplary method for dressing a wound may include applying a bandage having a top layer, a middle layer, and a bottom layer to a wound with the bottom layer being provided in contact with the wound, wherein the top layer includes an adhesive for securing the bandage to a surface near the would, and where the bottom layer includes copper; and allowing the bandage to remain over the would for a predetermined period of time. In some examples, the method may further include removing the bottom layer without removing the top and middle layers. In other examples, the method may include removing the bandage when the middle layer or the bottom layer change in color.

### CROSS-REFERENCE TO RELATED APPLICATION(S)

This application claims priority to U.S. Application No. 61/634,501, filed Feb. 29, 2012, and U.S. Application No. 61/686,539, filed Apr. 5, 2012, which provisional applications are incorporated herein by reference in their entirety for any purpose.

### TECHNICAL FIELD

Examples described herein relate generally to multi-layer bandages, and particularly to adhesive bandages and methods for dressing a wound.

### BACKGROUND

Silver has been used as an antibacterial agent for a long time, with various compositions of silver (e.g., silver nitrate, nanocrystalline silver, or others typically provided in colloidal form) being described and used in prior art bandages. However, while silver may be effective as an anti-biotic, it has also be determined to be toxic.

Copper, which has long been known to have health effect, has recently been rediscovered by scientists. Researchers have recently recognizing that dry copper kills tens to hundreds of millions of bacteria cells within minutes of contact by causing massive membrane damage, including anti-biotic resistant strains of bacteria, such as the so-called “flesh eating” bacteria, and even viruses like H1N1. Copper may be particularly effective because the bacteria and viruses do not survive contact, and thus cannot develop an immunity to copper over time.

### BRIEF DESCRIPTION OF THE DRAWINGS

The above and still further objectives, features, aspects and attendant advantages of the present invention will become apparent from the following detailed description of certain preferred and alternate embodiments and method of manufacture and use thereof constituting the best mode presently contemplated of practicing the invention, when taken in conjunction with the accompanying drawings, in which:

FIG. 1 is a schematic diagram of a multi-layer bandage according to an embodiment of the invention.

FIG. 2A is a side view of a multi-layer bandage according to an embodiment of the invention.

FIG. 2B is a side view of a multi-layer bandage according to another embodiment of the invention.

FIG. 2C is a side view of a multi-layer bandage according to another embodiment of the invention.

FIG. 3 is a top view of the embodiment of FIG. 2A

FIG. 4 is a side partially-open view of the embodiment of FIG. 2B.

FIG. 5 is a top-down view of a multi-layer bandage according to another embodiment of the invention.

FIG. 6. is a bottom-up view of the embodiment of FIG. 5 with release tabs (e.g., liner) closed to cover the contact portions of the bandage and adhesive.

FIG. 7 is a bottom-up view of the embodiment of FIG. 5 with the release tabs removed to expose adhesive and contact portion of the bandage.

FIG. 8 is a bottom-up view of the embodiment of FIG. 6 with the metallic layer removed and shown separately adjacent to the bandage.

### DETAILED DESCRIPTION

Certain details are set forth below to provide a sufficient understanding of embodiments of the invention. However, it will be appreciated by one skilled in the art that some embodiments may not include all details described. In some instances, well-known structures, materials, and processes have not been shown in order to avoid unnecessarily obscuring the described embodiments of the invention.

The present disclosure describes examples of multi-layer bandages and methods for dressing a wound. In some examples, the multi-layer bandage is configured in the size and/or shape of an adhesive bandage strip. Examples of a multi-layer bandage according to the present disclosure may include a first layer comprising a woven material including copper. In some examples, the woven material may be up to about 3 mil thick. In some examples, the woven material may be about 3 mil thick. In some examples, the woven material may be a woven polyester taffeta or other fabric wherein at least a portion of the polyester threads or strands are coated with copper. In some examples, the fabric may include copper of about 30% or more by weight. In some examples, the multi-layer bandage may contain up to about 30% by weight copper In some examples, copper content by weight may be between 20% and 45%. In some examples, pure copper may be used (e.g., a purity of up to about 99.999% copper). In some examples, the copper may have about 90% purity. Other purity and/or content by weight of copper may be used in various examples without departing from the scope of the present disclosure.

In some examples the bandage may be shaped to resemble a “pillow” shape, with a wicking material enclosed therein. Side tabs may be provided at two opposing side of the pillow-shaped structure for securing the bandage to the wound. In some examples, the wicking layer may be pure cotton gauze or other sanitary fiber material. The wicking layer may be adhered, using a non-toxic breathable glue or other non-toxic material, to a top layer, which may be similar to conventional backing layers of existing bandages. In some examples, the wicking layer may include synthetic or natural fibers. In some examples, the top layer be made of an air-permeable plastic layer. In some examples, the top layer may be a clear plastic layer (e.g., transparent layer). In some examples, the top and bottom sides of the pillow-shaped structure may be made of the same material, or they may comprise different materials (e.g., woven copper fabric at the bottom and a clear plastic material at top).

In some examples, the woven fabric may be copper taffeta fabric or other woven material using copper coated fibers. In some examples, the copper fabric of the bandage may be applied directly to an open wound to kill bacteria. The woven copper fabric may remain over the wound until the wound heals. In some examples, the bandage and or copper material may be removed sooner if so desired, and in some examples, configurations which allow the person to remove the copper material without undressing the wound are described. In some examples, the porous nature of the copper taffeta fabric, or other copper-based fabric, allows air to circulate through the wicking material and to the wound.

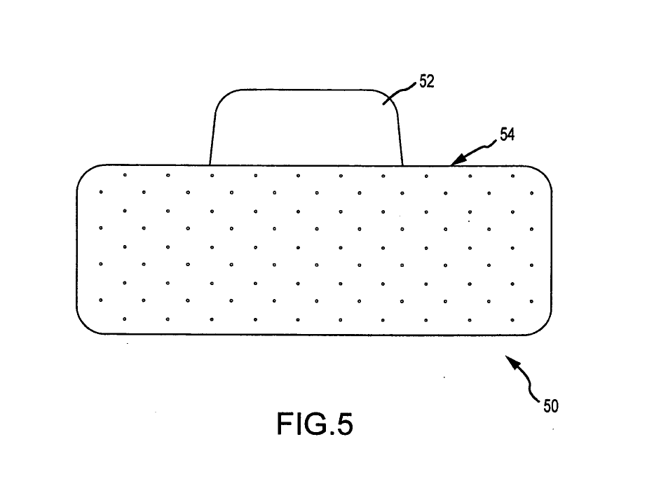

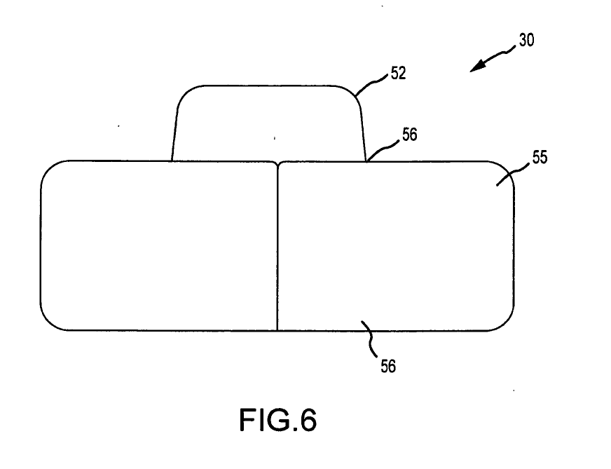

In some examples, in place of the woven material a metallic foil material is used as the first layer, which foil material may be removed as will be described further below with reference to FIGS. 5-8.

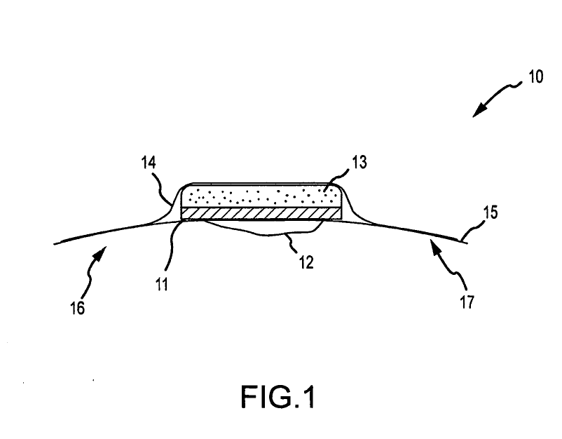

Referring now to the drawings, FIG. 1 is a schematic side view of a multi-layer bandage according to examples of the present disclosure. The multi-layer bandage 10 may include a first or metallic layer 11 for positioning directly in contact with the wound 12 and a second or wicking layer 13 disposed over the first layer 11 and configured to wick moisture away from the wound 12 through the first layer 11. In some examples, the bandage may include an optional third or attachment layer 14 which is adapted for affixing the bandage to the skin 15 of the person, for example by adhering the bandage 10 to the skin at locations 16, 17 adjacent to the wound 12.

The first layer 11 may be a fabric layer which includes copper. In some examples, the first layer 11 may be a woven fabric which includes a desired quantity of copper coated fibers. In some examples, the first layer 11 may be a woven fabric with about 35% by weight copper. In some examples, the first layer 11 may be a woven fabric having about 30% or more by weight copper. In some examples, the first layer 11 may contain up to about 30% by weight copper. In some examples, the first layer 11 may be a woven fabric having copper by weight in the range of about 25% to 50%. Other quantities of copper may be used. The fabric layer 11 may comprise synthetic or natural fibers, and in preferred examples, the fibers may be polyester fibers coated with dry copper. The fabric may be a woven fabric, for example a plain weave or taffeta weave fabric. In some examples other types of woven or knitted fabrics may be used.

The second or wicking layer 13 may include one or more sanitary fiber materials, for example pure cotton gauze. In some examples, other natural or synthetic fibers may be used, and blends of different fibers may be used as desired. The wicking layer may be provided as a knitted, woven or non-woven fabric. In some examples, cotton wool or other absorbent material may be used, as is frequently used in the medical industry. In some examples, the wicking layer may be gauze comprising pure cotton or other sanitary fibers. Other loosely woven or knitted materials may be used for facilitating the wicking of moisture from the wound.

In some examples, the optional third layer 14 may be provided over the first and second layers to secure the other two layers in place. In some examples, the third layer 14 may be attached to the other layers, or it may be laid over the wicking material to retain the wicking material and metallic material in place over the wound. The third layer 14 may include an adhesive portion 18, wherein any of a variety of adhesives generally used for similar type bandage may be provided for adhering the bandage to the subject. Other conventional methods of attaching and/or securing the bandage of the wound may be used for example, by using elastic or clasps. In further examples, the first 11 and third 14 layers may be made of the same material, for example, by weaving the pillow covering from the same copper based fibers. In some examples, a certain percentage of non-copper-coated fibers may be interspersed with the copper coated fibers.

In some examples, some or all of the third layer 14 may comprise a flexible material similar to the polymer backing material used in conventional bandages. In some examples, the third layer 14 may be transparent to enable observing the middle or wicking layer 12 therethrough. The third layer 14 may be air permeable to facilitate evaporation of the moisture collected in the wicking layer 12. In some examples, instead of using a transparent top layer, the third layer 14 may be provided with holes 27, which may serve the dual purpose of allowing air to permeate and circulate through the third layer 14 and further for observing the color of the middle layer 13 below.

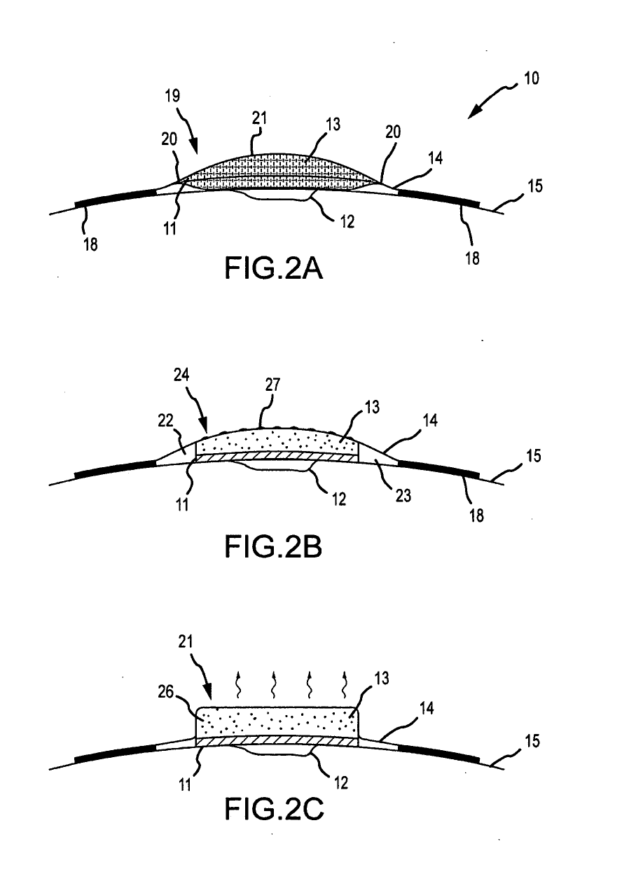

FIGS. 2A-2C show further examples of multi-layer bandages according to the present disclosure. Three configurations of multi-layer bandages are depicted, and other possible configurations may be implemented. FIG. 2A depicts a first configuration of a multi-layer bandage, which includes a first layer 11, and a second wicking layer 13. The bandage has a pillow-shaped structure 19. All sides of the pillow-shaped structure 19 may be formed using the same fabric material as used for the first layer 11. In some examples, a different material may be used for the top and bottom sides of the pillow-shaped structure 19. The third layer 14 may extend from two or more sides of the pillow-shaped structure 19 for securing the bandage in place. The third layer 14 may in some examples an attachment layer. The third layer 14 may be joined to the pillow-shaped structure 19 at edges 20 using non-toxic glue or sawn-in using sanitary fibers.

In some examples, the top of the bandage (e.g., top layer 21) may be an integral layer with the third layer 14. That is the attachment (e.g., top layer) may be a single material span the full length of the bandage 10, as shown in FIG. 2B. In other examples, and as shown in FIG. 2C, the top layer 21 and third layer 14 may be joined at the sides 21, 22. In some examples, the third layer 14 may be provided closer to the skin as in FIG. 2C and other examples, the third layer 14 may be spaced apart from the skin. Depending on the construction of the bandage 10 and spacing of various components, different amounts of pressure over the wound may advantageously be applied. In some examples, more pressure may be applied at the wound thereby helping to decrease the flow of fluids and serum out of the wound. In other examples, and based on the construction of the bandage, the wound may be more loosely dressed, to allow for increased circulation of may be implemented and appreciated in light of this disclosure.

FIG. 2B depicts another example of a multi-layer bandage according to the present disclosure. The top 21 and third layers are one and the same and the first layer 11 is not joined to the top layer but is instead secured to the wicking layer 13 using non-toxic materials (e.g., biocompatible non-toxic adhesive). The first layer 11 may be metallic, or include metallic materials. The first layer 11 may be a woven metallic material as well. In the example in FIG. 2B, the first layer 11 is again provided in direct contact with the wound 12, and the second or wicking layer 13 is disposed over the first layer 11. The wicking layer 13 may comprise a permeable material, and may be left open to the ambient air (see spaces 22, 23, and open sides 24 of the bandage) to facilitate wicking and evaporation of moisture from the wound 12. While the examples, in FIGS. 2B and 2C show an open-side configuration, the sides 24 of the multi-layer bandage 10 may be sealed as is depicted FIG. 2A, for examples. Thus, in some examples, permeability to the ambient air may be achieved through the top layer 21, which in this case is made of an air-permeable compliant material as described herein.

In some examples, and as shown in FIG. 1, and FIGS. 2B-2C, the wicking material and/or first layer 11 may have a generally rectangular profile in plan view. However, in some examples, a circular wicking pad may be used, and a correspondingly circular woven fabric layer may be adhered to the wicking pad and substantially covering the pad. In some examples, the wicking material may also have a rectangular profile in the transverse cross-sectional view (as seen in FIG. 1 and FIGS. 2B-2C). The wicking material 13 may be selected from a variety of materials and configured within the bandage such that it is able to substantially maintain its original shape, so as to provide sufficient spacing between the first layer 11 and the third layer 14. In this manner, the wicking layer 13 may act as a spacer 26 between the first layer 11 and top layer 21 to improve the ability of the wound to breathe. The space created by layer 13 may be particularly advantageous for drawing/wicking fluids from the wound trough the metallic layer. Furthermore, the first layer 11 which is provided between the wicking material 13 and the wound further acts as a separator between the wound 12 and the absorbent material 13, thereby further enhancing the wicking action allowing the wound to stay dry.

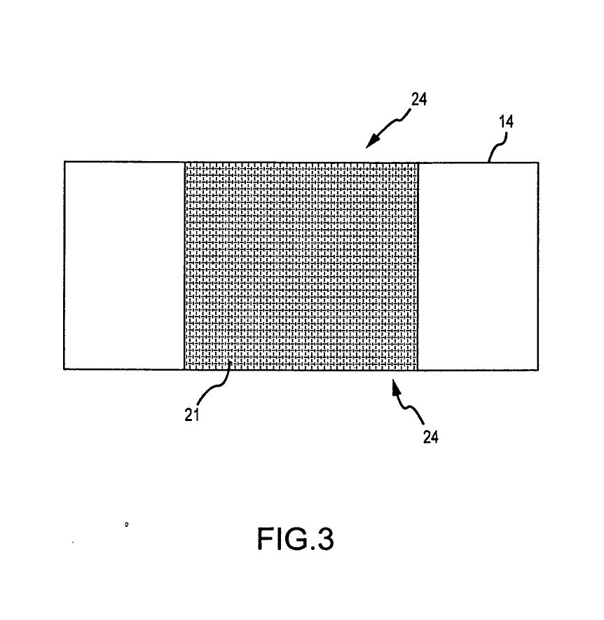

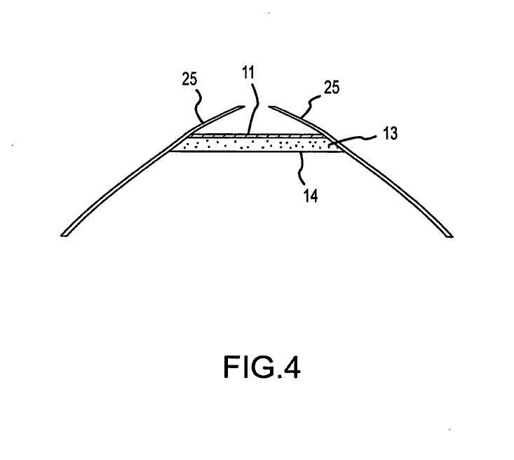

FIG. 3 shows a top view of the embodiment of the multi-layer bandage depicted in FIG. 2A. As will be appreciated, some embodiments may have similar features, and such features are numbered using the same numerals. Thus, for the sake of brevity their description and functionality will not be repeated. FIG. 4, shows a side view of a partially open multi-layer bandage of the example in FIG. 2B. The bandage 10 in FIG. 4 shows a pair of release tabs 25 that may be used to cover adhesive portions 18 of the third layer 14 until use.

Examples of methods for using a bandage according to the present disclosure and for dressing a wound are described herein. In some examples, a multi-layer bandage according to the present disclosure, for example a pillow style copper fabric adhesive bandage as shown in FIGS. 2A and 3, may be used according to the following procedure. The first layer of the bandage (e.g., the first layer 11) may be applied directly onto the wound 12. The bandage may, in some examples, be secured to the skin using the optional third layer 14, which may include side portions 18 with an adhesive or other conventional means for attachment of the bandage to the patient. In some examples, the bandage may remain in place over the wound for about 3 to 5 hours. In some examples, the bandage may be left over the wound 12 for less than 3 hours, or it may remain in place for 5 or more hours depending on the particular situation.

In some examples, the bandage 10 may be provided with a clear (e.g., transparent) top layer 21 to facilitate monitoring for the change of color of the layers below (e.g., layers 11 and 13). During the bacteria and/or virus neutralization process, while the copper layer is in contact with moisture and salts from the wound (e.g., serum or bodily fluids), the moisture and salts may interact with the copper causing the copper in the first layer 11 to begin turning green (e.g., verdigris formation). Upon observing a green copper verdigris color through the clear (e.g., transparent) bandage 10 the user may remove the bandage as the verdigris may indicate that the copper layer of the bandage has disinfected the wound. At this point the wound may be left open to air or a clean conventional bandage may be applied. In some example, verdigris may be observed through the clear upper layer 21 within 3 to 5 hours after the bandage is applied to the wound. In some examples, the bandage may be removed at any time prior to the copper layer turning green as may be desired.

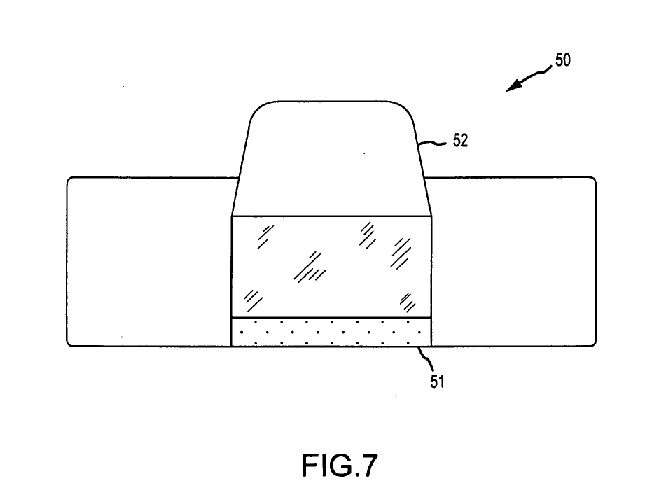

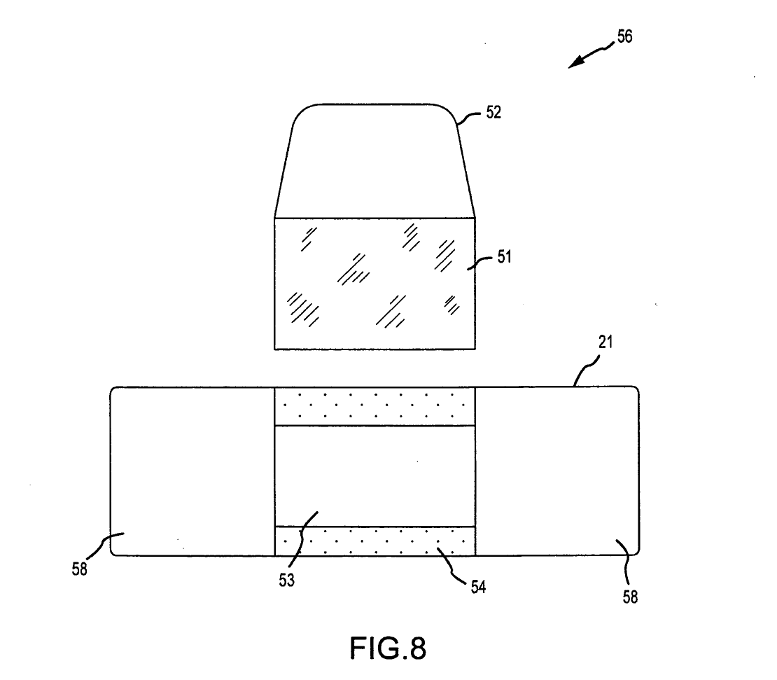

Referring now to FIGS. 5-8 another embodiment according to the present disclosure is described. The multi-layer bandage 50 in the example of FIGS. 5-8 includes a first or bottom layer 51, a second or middle layer 53, and a third or top layer 54. The first layer 51 may be a thin foil of metallic material, for example pure copper. In some examples, the layer 51 may include a pull tab 52 which may be attached to the layer 51 using a non-toxic material. In some examples, the layer 51 and pull tab 52 may be a unitary structure with the metallic material provided over a portion of the strip 56. Various conventional methods, such as vapor deposition or electroplating techniques, may be used to provide a thin layer of copper over a substrate 56. In other examples, the metallic material may be provided as a foil of the desired material (e.g., copper) and may be attached over a portion of the strip 56. In some example, and analogous to bandages configured as adhesive bandage strips, the multi-layer bandage 50 may be adapted to be secured to the skin of the patient, and accordingly, adhesive portions 58 may be provided on the top layer 54, and the adhesive portion may be covered using release tabs 55 placed over the adhesive portions 58 of the bandage while not in use. FIGS. 7 and 8 show a bottom-up view of bandage 50 with the first layer 51 exposed. During use, the layer 51, which may be pure copper having purity of up to about 99.999%, may be provided over the open wound. The bandage 50 is secured in place (e.g., to the patient's skin) using the adhesive portions 58. After a certain period of time, and without having to remove the bandage 50, the copper layer (e.g., layer 51) may be removed by pulling on the tab 52. The copper layer 51 is removed through the opening at the sides 56. The bandage 50 may remain in place for additional period of time allowing the middle layer 53, which may comprise any absorbent materials as described and known in the art, to wick moisture and aid with healing of the wound. The layer 51 may be adhered to the layer 53 using non-toxic, low tack (e.g., low strength) pressure sensitive adhesive so as to facilitate easy removal of the layer 51 using the pull tab 52. In some examples, a light adhesive, such as the 3M Company paper memo pad glue, may be used.

There may be numerous advantages of the present disclosure as described herein and as will be appreciated in view of the examples described herein. Thin copper coated polyester fabric pillow-style adhesive bandages can be a highly effective, low cost antibiotic first-aid response application for minor wound care, and for more serious infections. For example, the cost for 1.24 square inches (enough for the top and bottom of one small pillow-style adhesive bandage) of 3 mil thick 35% pure copper coated polyester taffeta fabric may be minimal, (e.g., roughly about two U.S. cents at retail as of May 8, 2012). This amount may be sufficient for one complete bandage. Furthermore, examples of the technologies described herein may be readily adaptable to current adhesive bandage production with little added expense, other than the fabrication of pillow-shaped copper structures enclosing the wicking layer therein. Additional benefit may be obtained by the wicking effect which be effectively achieved by the combination of a woven porous copper layer separating the wicking layer from the wound. That is, in some examples, cotton gauze may absorb the fluids from the wound through the copper taffeta fabric via capillary action, and the copper taffeta fabric then maintains a barrier between the now wet cotton gauze and the wound, effectively drying the wound, which further facilitation the wound to heal faster.

Although examples of the invention have been described herein, it will be recognized by those skilled in the art to which the invention pertains from a consideration of the foregoing description of presently preferred and alternate embodiments and methods of fabrication and use thereof, that variations and modifications of this exemplary embodiment and method may be made without departing from the true spirit and scope of the invention. Thus, the above-described embodiments of the invention should not be viewed as exhaustive or as limiting the invention to the precise configurations or techniques disclosed. Rather, it is intended that the invention shall be limited only by the appended claims and the rules and principles of applicable law.

What is claimed is:

1. A multi-layer bandage comprising:
a first layer comprising a woven material including copper and configured to be in direct contact with an open wound; and
a second layer adjacent the first layer, the second layer including a wicking material configured to draw moisture from the wound thought the first layer.

2. The multi-layer bandage of claim 1, wherein the woven material comprises 30% or more by weight copper.

3. The multi-layer bandage of claim 1, wherein the woven material comprises up to 30% by weight copper.

4. The multi-layer bandage of claim 2, wherein the woven material comprises copper coated fibers, the copper having greater than 90% purity.

5. The multi-layer bandage of claim 1, wherein the woven material has a thickness of up to about 3 mil.

6. The multi-layer bandage of claim 1, wherein the wicking material comprises cotton gauze.

7. The multi-layer bandage of claim 1, further comprising a third layer disposed over at least a portion of the second layer and adapted for securing the multi-layer bandage to a subject

8. The multi-layer bandage of claim 7, wherein the third layer comprises an air-permeable compliant substrate having adhesive provided on a surface thereof.

9. The multi-layer bandage of claim 7, wherein the second layer is fully enclosed between the first and third layers.

10. The multi-layer bandage of claim 7, wherein the third layer is configured to allow a change in color of the second layer or the first layer to be observed through the third layer.

11. The multi-layer bandage of claim 7, wherein the third layer is configured to allow evaporation of moisture from the second layer to the ambiance.

12. A multi-layer bandage comprising:
a top layer including an adhesive applied to a portion of the top layer;
a middle layer including an absorbent pad and provided adjacent to the top layer; and
a bottom layer configured to be provided in direct contact with a wound, the bottom layer being removably attached to at least one of the middle layer or top layer such that the bottom layer is removable without removing the middle or top layers of the bandage.

13. The multi-layer bandage of claim 12, wherein the bottom layer comprises a metal foil having a pull tab, the metal foil being removably attached to the middle layer using a non-toxic material

14. The multi-layer bandage of claim 12, wherein the metal foil comprises copper having about 90% or more purity.

15. The multi-layer bandage of claim 12, wherein the third layer is attached to the second layer using non-toxic adhesive.

16. The multi-layer bandage of claim 12, wherein the third layer has a thickness of about 1 mil or greater.

17. A method of dressing a wound comprising:
providing a contact layer of a multi-layer bandage in contact with a wound, wherein the multi-layer bandage further includes a wicking layer adjacent the contact layer and an attachment layer configured for securing the contact and wicking layers over the wound;
removing the multi-layer bandage when the wicking layer or the contact layer change in color.

18. A method of dressing a wound comprising:
applying a bandage having a top layer, a middle layer, and a bottom layer to a wound with the bottom layer being provided in contact with the wound, wherein the top layer includes an adhesive for securing the bandage to a surface near the wound, and wherein the bottom layer includes copper; and
removing the bottom layer without removing the top and middle layers.
